(19) United States
(12) Patent Application Publication
(10) Pub. No.: US 2026/0144939 A1
(43) Pub. Date: May 28, 2026

(54) MULTI-DOSE SYRINGE AND METHOD OF MULTIPLE DOSING A LIQUID OR SEMILIQUID PRODUCT

(71) Applicant: PLATINUM PHARMA SERVICE S.R.L.S., Città Sant'Angelo (IT)

(72) Inventor: Alfredo RICCI, Città Sant'Angelo (IT)

(73) Assignee: PLATINUM PHARMA SERVICE S.R.L.S., Città Sant'Angelo (IT)

(21) Appl. No.: 18/718,868

(22) PCT Filed: Dec. 5, 2022
(86) PCT No.: PCT/IB2022/061774

§ 371 Date: Jun. 12, 2024

(30) Foreign Application Priority Data
Dec. 16, 2021 (IT) 102021000031520

(51) Int. Cl. A61M 5/315; A61D 7/00; A61M 5/31

(52) U.S. Cl. CPC A61M 5/3153; A61D 7/00; A61M 5/3155; A61M 5/3156; A61M 2005/3106

(57) ABSTRACT

A multi-dose syringe and a method of multiple dosing a product. The syringe includes a hollow cylindrical body containing a product to be administered in a dosed manner and from which a nozzle extends provided with a product outlet opening, and a plunger, sliding within the hollow cylindrical body. A helical indentation is formed on an outer surface of the plunger, which includes a plurality of teeth, each having a first and second longitudinal surface and a first and second transverse surface extending, parallel to each other, orthogonally from a respective longitudinal surface and further including a flanged dose-setting sleeve, irremovably mounted on one end of the hollow cylindrical body for the entry of the plunger, and a dose-setting ring mounted on the flanged dose-setting sleeve and rotatable with respect thereto and with respect to the plunger.

### TECHNICAL FIELD

The present invention generally relates to the technical field of dosing syringes. More particularly, the present invention relates to a syringe, preferably for veterinary use, configured to administer precise and repeatable doses of a liquid or semiliquid product over time, for example a medicine or dietary supplement, contained therein, and a method of multiple dosing a product. The multi-dose syringe according to the present invention finds further application in the food sector, or in any other sector where it is necessary to administer multiple, repeatable doses of the same liquid or semiliquid product.

### BACKGROUND

Dosing syringes, of the pre-filled or refillable type, are widely used in the veterinary field for feeding animals, especially pets, but not only.

They represent a growing market, as they effectively reduce errors in dosing a product to be administered. In fact, once filled, or purchased pre-filled with the desired product, such as a medicine or dietary supplement, they can easily be used to dose, and subsequently administer, the correct dose for a specific animal.

As is well known, in the veterinary field, the dosage of a medicine or dietary supplement to be administered to an animal varies considerably depending on the animal's weight.

Disposable syringes are usually used to administer a dose of the product to an individual animal. In this case, the veterinarian selects the appropriate dose on the syringe for the particular animal to be treated, dispenses the selected dose and disposes of the syringe with the remaining product content inside. Alternatively, when a product is to be administered to a large number of animals, multiple doses can be contained in a single syringe and the veterinarian selects the appropriate dose for the particular animal to be treated on the syringe and dispenses the selected dose.

It follows that dosing syringes must be capable of administering doses of varying amounts, consistent with the weight of the animal to be treated.

A dispensing syringe according to the prior art is described, for example, in international patent application no. WO 02/26298 A2. This dosing syringe comprises a cylindrical body containing a product to be administered in doses. The cylindrical body has an opening at one end for dispensing a dose of product, while the other end is adapted to receive a plunger that is longer than the length of the cylindrical body and slides inside the cylindrical body. The plunger has a series of indentations along its entire length. The dispensing syringe also includes a movable dose selection ring, which is arranged around the plunger and can be rotated about the longitudinal axis of the plunger. In particular, the ring is screwed around the plunger, as the indentations on the plunger form a kind of thread. The movable dose selection ring comprises calibration means, formed on a sleeve and adapted to set the desired dose by rotating the movable ring until the desired dose is reached, and means of engaging the movable ring with the plunger, such that the movable ring moves integrally with the plunger until it abuts against the cylindrical body of the syringe, so as to dispense the desired set dose.

However, known dispensing syringes of the type described above have the following drawbacks.

Firstly, whenever the user wishes to administer a dose, he must set it by acting on the movable ring, i.e. turning it along a thread, as briefly described above. This is particularly disadvantageous when the dose to be administered is always the same, e.g. in the case of administering equal doses of a medicine to the same animal at different times of the day or in the case of administering the same dose to different animals with the same or similar body weight. In this case, in addition to performing an unnecessary operation, the operator also runs the risk of making a mistake in setting the dose. In addition, the longitudinal positioning of the movable ring by means of a thread is a time-consuming operation.

Secondly, when setting a dose, the animal may move, causing an unwanted rotation of the movable dose-setting ring during the administration of the product. This is disadvantageous as it would compromise the accuracy of the previously set dose. In fact, each rotation of the movable ring corresponds to a different dose to be administered.

Other dosing syringes known in the state of the art involve an insert, which inserts/disconnects into/from the plunger, with similar problems to those described above.

### OBJECTS AND SUMMARY OF THE INVENTION

The main aim of the present invention is therefore that of making available a multi-dose syringe and method of multiple dosing a liquid or semiliquid product that can overcome the drawbacks mentioned above with reference to dosing syringes of the known type.

More specifically, the main purpose of the present invention is to make available a multi-dose syringe and a method for multiple dosing of a liquid or semiliquid product, such that precise and repeatable doses of the product contained in the syringe or even doses with different volumes can be administered.

Another purpose of the present invention is to make available a multi-dose syringe and a method of multiple dosing a liquid or semiliquid product, such as to enable the dose to be administered to be varied quickly and immediately.

Yet another purpose of the present invention is to make available a multi-dose syringe and method of multiple dosing a liquid or semiliquid product, such that dosing errors are avoided, either by the user or due to external causes unrelated to the user, e.g. impact with an animal or other stresses.

Yet another purpose of the present invention is to make available a multi-dose syringe, configured in such a way as to avoid loss of time in the dose-setting phase, whether they are doses with constant volumes or doses with variable volumes.

Yet another purpose of the present invention is to make available a multi-dose syringe that is structurally simple and easily assembled.

Last but not least, the purposes of the present invention is to make available a multi-dose syringe, which can be produced with competitive times and costs.

These and other objects of the present invention are achieved by a multi-dose syringe and a method of multiple dosing a liquid or semiliquid product incorporating the features of the accompanying claims, which form an integral part of the present description.

The invention thus relates, in a first aspect thereof, to a multi-dose syringe comprising a hollow cylindrical body, containing a product to be administered in a dosed manner and from which a nozzle extends provided with a product outlet opening, and a plunger, sliding within the hollow cylindrical body.

A helical indentation is formed on an outer surface of the plunger, which comprises a plurality of teeth, each having a first and a second longitudinal surface and a first and a second transverse surface, extending, parallel to each other, orthogonally from a respective longitudinal surface and further comprising a flanged dose-setting sleeve, irremovably mounted on one end of the hollow cylindrical body for the entry of the plunger, and a dose-setting ring mounted on the flanged dose-setting sleeve and rotatable with respect thereto and with respect to the plunger.

A series of seats and at least one tooth configured to be housed with interference into a respective seat are provided on the facing surfaces of the flanged sleeve and ring, respectively.

The plunger can be rotated with respect to the hollow cylindrical body until the first longitudinal surface of a tooth abuts against a ring abutment surface projecting towards the inside of the hollow cylindrical body, in order to set a predetermined dose of the product to be administered.

Due to such a combination of features, in particular due to the aforementioned interference coupling between the flanged dose-setting sleeve and the dose-setting ring and the interaction of the helical indentation of the plunger with the dose-setting ring, the syringe according to the invention allows for simple and immediate administration of predetermined and repeated doses of a product. Furthermore, once the dose to be administered has been set, it cannot be changed accidentally, e.g. due to the movement of an animal, but only the user can vary the dose if desired.

In one embodiment, the flanged dose-setting sleeve comprises a first annular body, mounting the flanged sleeve on the end of the hollow cylindrical body for the entry of the plunger, a second annular body provided with an opening and a flange interposed between the first annular body and the second annular body, the housing seats with interference of at least one tooth of the dose-setting ring being formed on a surface of the flange.

In one embodiment, there is a stop element on an inner surface of the second annular body, against which the first transverse surface of a tooth of the helical indentation abuts, in order to interrupt the stroke of the plunger within the hollow cylindrical body, when dispensing the dosed amount of product.

In one embodiment, the plunger comprises a rubber stopper associated with one end of the plunger intended to slide within the hollow cylindrical body, a connector being interposed between the rubber stopper and the end of the plunger to allow for rotation of the plunger relative to the rubber stopper within the hollow cylindrical body.

In one embodiment, the dose-setting ring comprises a main body, from which it extends towards the outside of a tab, the interference housing seats of at least one tooth of the dose-setting ring being formed at one surface of the tab.

In one embodiment, the tab extends outwards from a portion of the dose-setting ring, which has a reduced thickness, preferably comparable to the thickness of the tab. The tab is therefore, advantageously, more flexible and therefore easier for the user to handle when setting a dose of product to be administered.

In one embodiment, the dose-setting ring includes at least one, preferably two recesses, of localised weakening of the dose-setting ring.

In one embodiment, the abutment surface of a tooth of the plunger indentation is formed on an extension of the tab facing towards the inside of the dose-setting ring.

In one embodiment, a stop extends from a flange surface facing the second annular body to limit the rotation of the dose-setting ring with respect to the flanged dose-setting sleeve or to exclude the dose-setting ring from cooperation with the flanged dose-setting sleeve.

In one embodiment, progressive numbers, one for each seat, are marked at the flanged sleeve seats, each indicating a quantity, preferably expressed in millilitres, of product to be dosed and subsequently administered in a dosed manner.

In one embodiment, there is a handle at one end of the plunger, preferably with a faceted outer surface.

In one embodiment, the nozzle is preferably made in one piece with the hollow cylindrical body, or is a separate piece subsequently assembled on the hollow cylindrical body, and is preferably closed with a cap. Making the nozzle as a separate piece has the advantage of reducing the dimensions of the multi-dose syringe during transport and consequently shipping and packaging costs. Furthermore, a multi-dose syringe without a nozzle can be advantageously pre-filled using any known filling machine.

In a second aspect, the invention relates to a method of multiple dosing a product performed using the multi-dose syringe defined above, the method comprising the following steps:
rotation of a dose-setting ring with respect to a flanged dose-setting sleeve irremovably mounted on one end of a hollow cylindrical body of the multi-dose syringe until at least one tooth of the ring is positioned at a respective seat of the flanged sleeve, corresponding to a predetermined volume of product to be dosed;
interference coupling between tooth and seat;
rotation of a plunger with respect to the flanged sleeve—dose-setting ring assembly, until a first longitudinal surface of a tooth of a helical indentation formed on an outer surface of the plunger abuts against a ring abutment surface, projecting into the dose-setting ring, so as to set a predetermined dose of the product to be administered.

In one embodiment, the method further comprises, downstream of the plunger rotation step, a step of pressing the plunger towards the inside of the hollow cylindrical body, until a second transverse surface of a tooth of the helical indentation abuts against a stop element in the flanged sleeve, so that the plunger's stroke inside the hollow cylindrical body is interrupted when the dosed amount of product is administered.

In one embodiment, the method comprises the following further steps of setting a new dose of product to be administered:
lifting the ring from the flanged sleeve, so that the tooth is released from its seat;
rotation of the ring with respect to the flanged sleeve until at least one tooth of the ring is positioned at a respective seat of the flanged sleeve corresponding to a new dose to be administered;
interference coupling between the tooth and the seat; and
rotation of a plunger with respect to the flanged sleeve—ring assembly, until a first longitudinal surface of a tooth of a helical indentation of the plunger abuts against the ring abutment surface, projecting into the dose-setting ring, so as to set a predetermined dose of the product to be administered.

Further features and advantages of the present invention will be more evident from the description of the accompanying drawings.

### BRIEF DESCRIPTION OF THE DRAWINGS

The invention is described hereinbelow with reference to certain examples provided by way of non-limiting example and illustrated in the accompanying drawings. These drawings illustrate different aspects and embodiments of the present invention and reference numerals illustrating structures, components, materials and/or similar elements in different drawings are indicated by similar reference numerals, where appropriate.

FIG. 1 is a perspective view of a multi-dose syringe according to a first preferred embodiment of the present invention;

FIG. 2 is an exploded perspective view of the multi-dose syringe of FIG. 1;

FIG. 3 is a longitudinal section view of the multi-dose syringe of FIG. 1;

FIG. 4 is a partial perspective view of a plunger of the multi-dose syringe of FIG. 1;

FIG. 5 is a perspective view, taken from another angle, of the plunger of FIG. 4;

FIG. 5A is an enlarged view of the detail circled in FIG. 5;

FIG. 6 is a perspective view of a flanged dose-setting sleeve of the multi-dose syringe of FIG. 1;

FIG. 7 is a perspective view from above of a dose-setting ring of the multi-dose syringe of FIG. 1;

FIG. 8 is a perspective view from below of the dose-setting ring of FIG. 7;

FIG. 9 is a perspective view from above of an alternative embodiment of a dose-setting ring of the multi-dose syringe of FIG. 1;

FIG. 10 is a perspective view from below of the dose-setting ring of FIG. 9;

FIG. 11 is a partial perspective view of the multi-dose syringe of FIG. 1, in an operational step in which the dose to be administered has been set and the plunger is about to be rotated to prepare the syringe for dispensing the set dose;

FIG. 12 is a partial perspective view of the multi-dose syringe of FIG. 1, showing an operational step in which the dose to be administered has been set, the plunger has been rotated and is ready to be pressed to allow the previously set product dose to be administered;

FIG. 13 is a partial perspective view of the multi-dose syringe of FIG. 1, showing the multi-dose syringe of FIG. 1 in the step of completing the administration of a pre-set dose; and

FIG. 14 is a partial perspective view of the multi-dose syringe of FIG. 1, showing an operational step in which the dose-setting ring is excluded from cooperation with the flanged dose-setting sleeve and the plunger can rotate freely to deliver a measured, and not pre-set, dose of liquid or semiliquid product.

### DETAILED DESCRIPTION OF THE INVENTION

While the invention is susceptible to various modifications and alternative constructions, certain preferred embodiments are shown in the drawings and are described hereinbelow in detail. It must in any case be understood that there is no intention to limit the invention to the specific embodiment illustrated, but, on the contrary, the invention intends covering all the modifications, alternative and equivalent constructions that fall within the scope of the invention as defined in the claims.

The use of “for example”, “etc.”, “or” indicates non-exclusive alternatives without limitation, unless otherwise indicated. The use of “includes” means “includes, but not limited to” unless otherwise indicated.

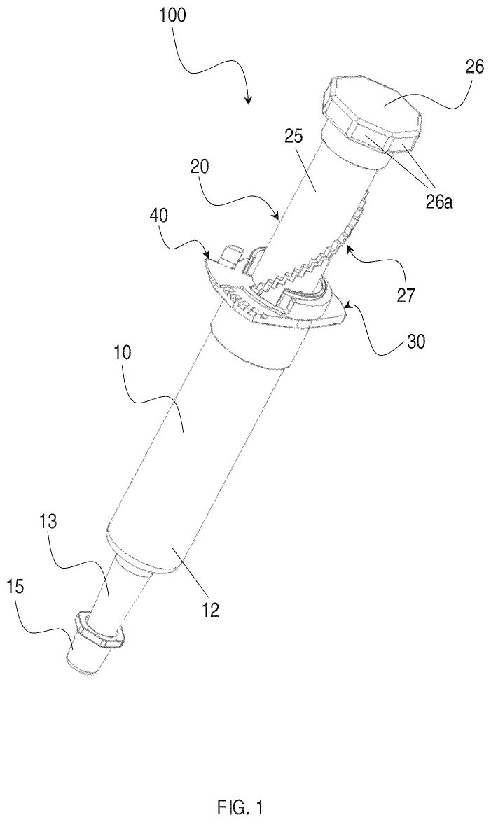

With reference to FIGS. 1 to 3, they show a multi-dose syringe according to a preferred embodiment of the present invention.

The multi-dose syringe, generally referred to by the reference number 100, is preferably a pre-filled syringe, but it may also be filled at the time of administration, and comprises a hollow cylindrical body or barrel 10, either transparent or opaque, containing a liquid or semiliquid product, e.g. a medicine or dietary supplement, to be dosed and administered to an animal, for example.

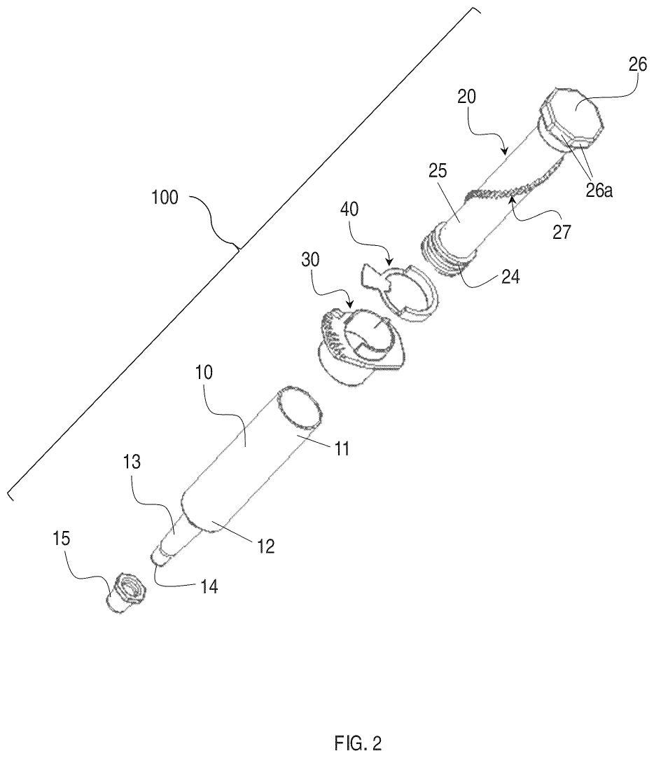

As can be seen in FIG. 2, the hollow cylindrical body 10 has an end 11 for insertion of a plunger 20 and another end 12, opposite the end 11 for insertion of the plunger 20, from which a nozzle 13 extends, hollow and substantially truncated, provided with an opening 14 for the outlet of the product being administered.

The nozzle 13 is preferably made in one piece with a hollow cylindrical body 10 and is preferably closed with a cap 15. In an alternative embodiment, the nozzle 13 is a separate element, which is assembled on the hollow cylindrical body 10 when the dosing syringe 100 is used. This advantageously saves on packaging material and reduces space during storage and transport of the multi-dose syringe.

As will be described in detail below, the plunger 20 is configured to rotate about a longitudinal axis of the multi-dose syringe 100 and to slide inside the hollow cylindrical body 10, respectively, to set a dose of the product contained in the syringe and subsequently administer the set dose in a precise and repeatable manner over time.

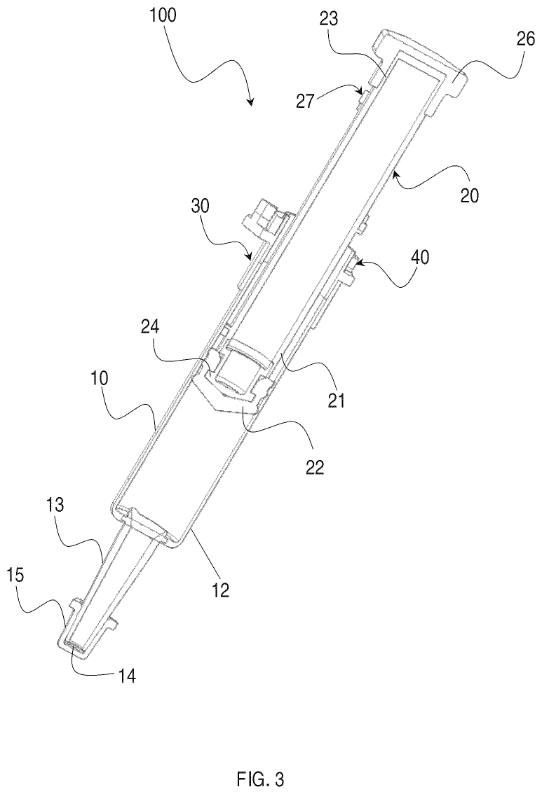

As can be seen in detail in FIG. 3, the plunger 20 consists of a hollow cylinder, which at one end 21 bears a rubber stopper 22 intended to slide inside the hollow cylindrical body 10 of the syringe 100. When the plunger 20 is pressed by the user to administer a dosed amount of product, the rubber stopper 22 compresses the product, while at the same time creating a seal with an inner surface of the hollow cylindrical body 10. In other words, the rubber stopper 22 ensures that, during administration, the product contained in the hollow cylindrical body 10 of the multi-dose syringe 100 is directed towards the product outlet nozzle 13 and not in the opposite direction.

Preferably, a connector 24 is interposed between the end 21 of the plunger 20 and the rubber stopper 22, the function of which, as will be described in greater detail below, is to allow a rotation of the plunger 20 with respect to the rubber stopper 22, while ensuring the fluid seal as the plunger 20 slides within the hollow cylindrical body 10 of the multi-dose syringe 100. The connector 24 can be a separate component, mounted on the 21 end of the plunger 20, or it can be made as a single piece with the plunger 20.

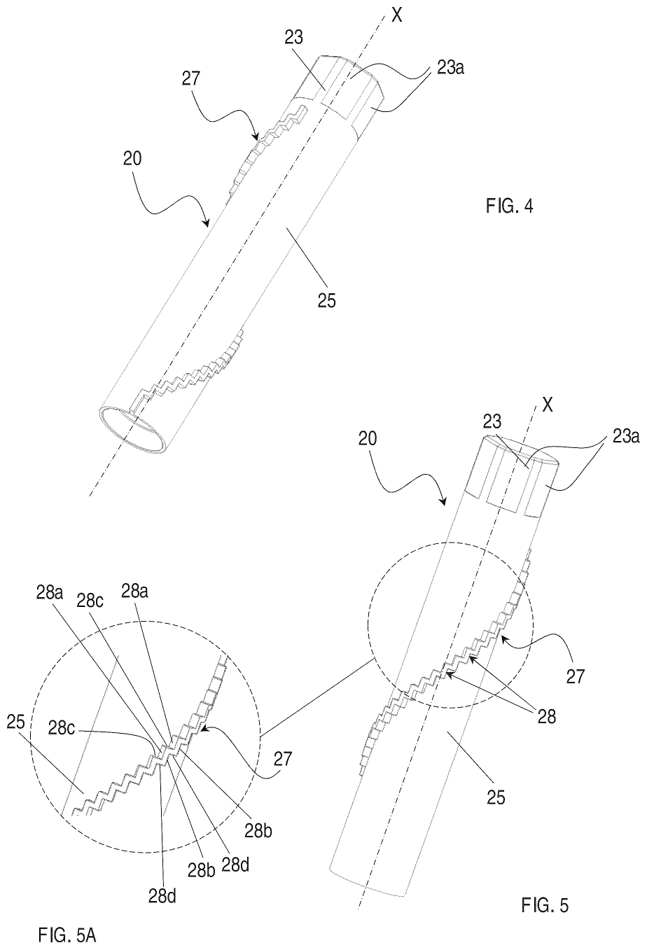

At another end 23 of the plunger 20, opposite the end 21 bearing the rubber stopper 22, there is a handle 26, on which the user acts with his thumb to push the plunger 20 into the hollow cylindrical body 10 when administering a pre-set dose of product. In the illustrated embodiment, the handle 26 is a separate element, which is mounted on the end 23 of the plunger 20, permanently and without any relative movement between the handle 26 and the plunger 20. For this purpose, and as shown in detail in FIGS. 4 and 5, the end 23 is faceted, with a number of flat faces 23a corresponding to the number of flat faces (not shown) obtained at an inner surface of the handle 26.

Preferably, the handle 26 has a faceted outer surface comprising a number of flat faces 26a (FIGS. 1 and 2), e.g. eight flat faces 26a. This faceted outer surface advantageously allows for a better grip of the handle 26 by the user.

With reference to FIGS. 4, 5 and 5A, on an outer surface 25 of the plunger 20 a helical indentation 27 is formed, comprising a plurality of teeth 28, which extends over the entire outer surface 25 or, alternatively, over part of it. The teeth 28 have a similar pattern to that of a spiral staircase.

As shown in detail in FIG. 5A, each tooth 28 of the helical indentation 27 has a first longitudinal surface 28a and a second longitudinal surface 28b, which extend, parallel to each other, along a longitudinal axis X (shown for example in FIG. 5) of the plunger 20, and a first transverse surface 28c and a second transverse surface 28d, which extend, parallel to each other, orthogonally from a respective longitudinal surface 28a, 28b, to form a respective tooth 28 of the helical indentation 27. The first 28c and second transverse surface 28d of a tooth 28 positioned upstream, according to a direction from the coupling end 23 with the handle 26 to the coupling end 21 with the manifold 24, are connected to the first 28a and second longitudinal surface 28b of a tooth 28 positioned downstream according to the same direction.

Referring again to FIGS. 1 to 3, the multi-dose syringe 100 also comprises a flanged dose-setting sleeve 30 and a dose-setting ring 40, which cooperate with each other, and with the helical indentation 27, to set, and subsequently administer, the desired dose of product.

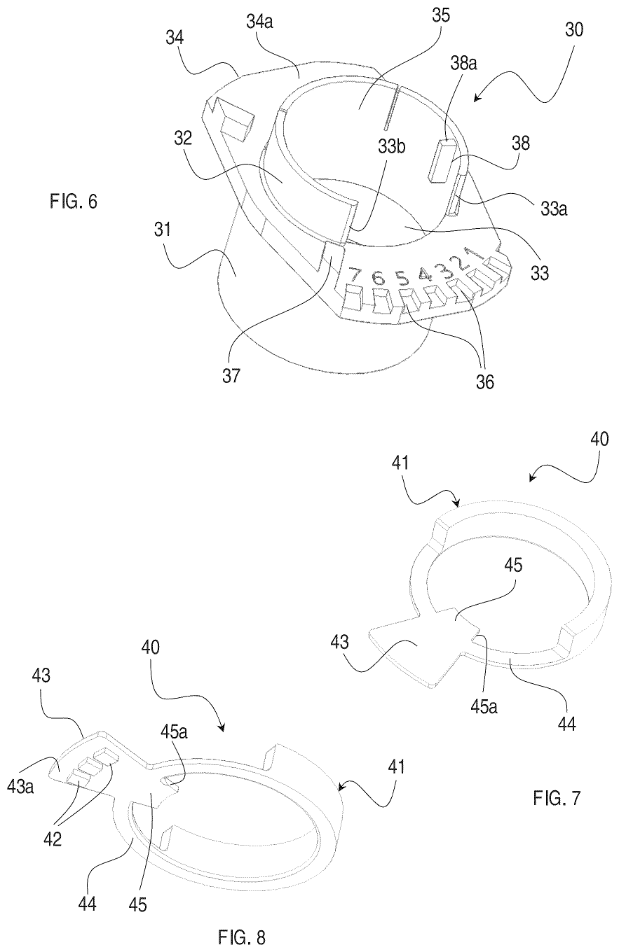

In particular, and as shown in detail in FIG. 6, the flanged sleeve 30 comprises a first annular body 31 and a second annular body 32 and a flange 34 interposed between the first annular body 31 and the second annular body 32. The second annular body 32 is provided with an opening 33 and is preferably smaller in diameter than the first annular body 31.

The first annular body 31 is adapted to be fitted irremovably onto the end 11 of the hollow cylindrical body 10 of the multi-dose syringe 100 and the dose-setting ring 40 is adapted to be rotatably mounted on the flanged sleeve 30, more particularly, outside the second annular body 32.

On an internal surface 35 of the second annular body 32, in a position suitably designed for obtaining the desired preset doses, specifically in proximity to a side wall 33a of the opening 33, there is a stop element 38, the function of which is to interrupt the stroke of the plunger 20 within the hollow cylindrical body 10, during the step of administering the dosed amount of product.

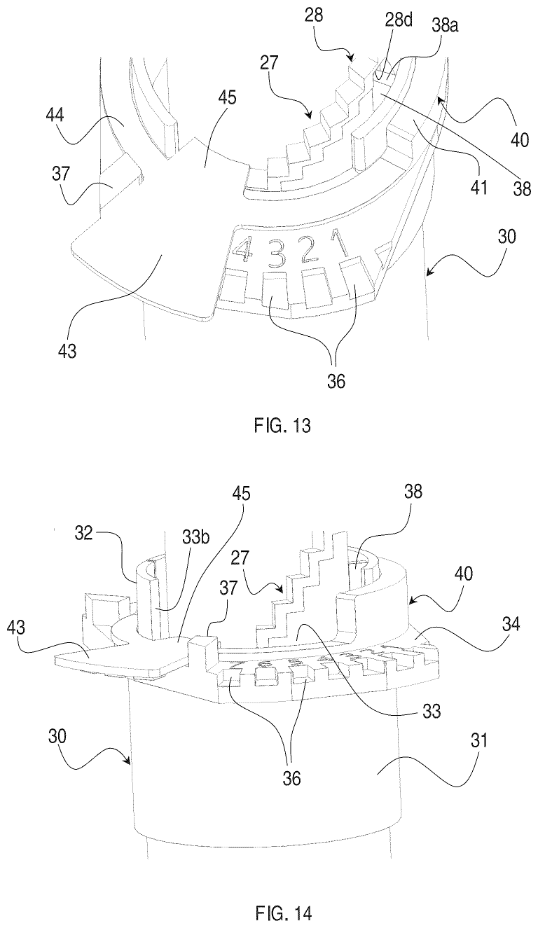

Furthermore, a stop 37 extends from a surface 34a of the flange 34 facing the second annular body 32, precisely in proximity to a side wall 33b of the opening 33, opposite the side wall 33a, whose function is to limit the rotation of the dose-setting ring 40 with respect to the flanged dose-setting sleeve 30, thereby determining the maximum amount of product that can be administered. In addition, and as will be described below with reference to FIG. 13, the stop 37 allows the dose-setting ring 40 to be excluded from cooperation with the flanged dose-setting sleeve 30, so that the plunger 20 can rotate freely to administer a desired, and not pre-set, dose of liquid or semiliquid product.

On the surface 34a of the flange 34, there is also a series of interference housing seats 36 for one or more teeth 42 (see FIG. 8) formed in the dose-setting ring 40. If the number of teeth 42 exceeds one, they are preferably arranged like an arc of a circle, the centre of which coincides with the longitudinal axis of the multi-dose syringe 100. Thanks to the interference coupling between the seats 36 and the teeth 42, any relative movement between teeth 42 and seats 36 is advantageously prevented, so that the user can set the dose to be dispensed in a way that cannot be altered unintentionally, e.g. as a result of an accidental shock to the multi-dose syringe 100, e.g. by the animal to which the dose is to be administered.

Furthermore, and as will be set out in detail later in this description, the purpose of the seats 36 in the flanged sleeve 30 and the tooth/teeth 42 in the ring 40 is to engage with each other, thereby uniquely and repeatably determining the dose of product to be administered. To this end, progressive numbers, one for each seat, are preferably shown at the seats 36 of the flanged sleeve 30, in FIG. 6 the numbers 1 to 7, each indicating a quantity, preferably expressed in millilitres, of product to be dosed and subsequently administered. Each seat 36 thus corresponds to a different, predetermined dose of product to be administered, which, once set by coupling between the dose-setting ring 40 and the flanged dose-setting sleeve 30, remains fixed and can thus be administered precisely and repeatably over time.

As shown in detail in FIGS. 7 and 8, the dose-setting ring 40 comprises a main body 41, from which a tab 43 extends, which is movable within the opening 33 of the flanged sleeve 30. At a surface 43a of the tab 43 facing the surface 34a of the flange 34 of the flanged sleeve 30 during use, teeth 42 are formed, which, as set forth above, are configured to be accommodated, with interference, within the respective seats 36 formed on the surface 34a of the flange 34, to set a predetermined and repeatable dose of product to be administered.

Preferably, the ring 40 comprises a portion 44, precisely the portion from which the tab 43 extends outwards, which is thinner, preferably flat and therefore less rigid. This advantageously allows the user to easily lift, by elastically deforming it, the tab 43 to release the teeth 42 from their respective seats 36, if, for example, the dose of product to be administered in a repeatable manner over time is to be changed.

An extension 45 of the tab 43 also extends towards the inside of the dose-setting ring 40 to create an abutment surface 45a for the teeth 28 of the helical indentation 27 formed on the outer surface 25 of the plunger 20.

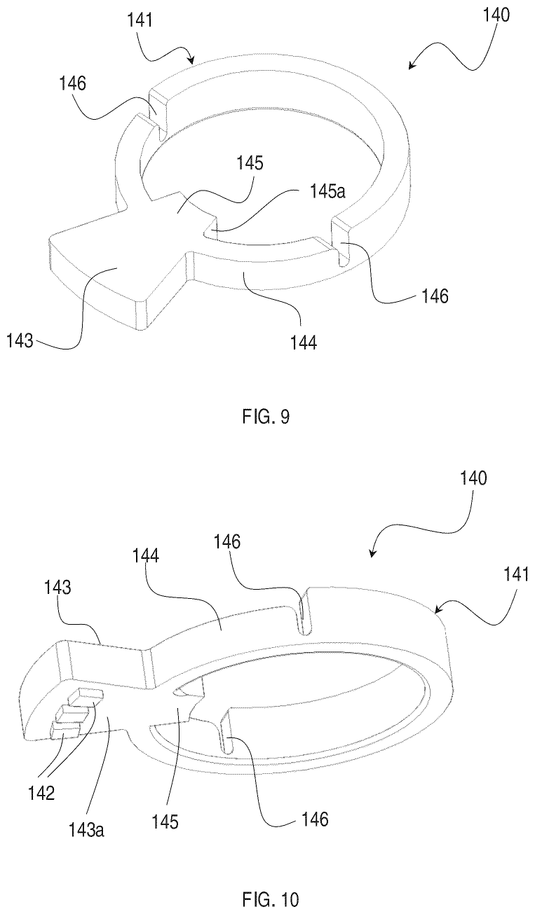

FIGS. 9 and 10 illustrate an alternative embodiment of a dose-setting ring of the multi-dose syringe 100 according to the present invention. The dose-setting ring, generally indicated by reference number 140, differs from the dose-setting ring 40 described above with reference to FIGS. 7 and 8, in that it provides, instead of a substantially flat portion, at least one, preferably a pair of recesses 146.

The dose-setting ring 140 thus comprises a main body 141, from which a tab 143 extends, which is movable within the opening 33 of the flanged sleeve 30. At a surface 143a of the tab 143 facing the surface 34a of the flange 34 of the flanged sleeve 30 during use, teeth 142 are formed, which are intended to be accommodated, with interference, within the respective seats 36 formed on the surface 34a of the flange 34, to set a predetermined and repeatable dose of product to be administered.

Preferably, the ring 140 comprises a portion 144, precisely the portion from which the tab 143 extends outwards, which is slightly thinner. In addition, in the transition area between the main body 141 and the portion 144, recesses 146 are created. The recesses 146 constitute localised weakening zones of the ring 140 and therefore advantageously allow the user to easily lift the tab 143 to release the teeth 142 from their respective seats 36, if, for example, the dose of product to be administered in a repeatable manner over time is to be changed.

An extension 145 of the tab 143 also extends towards the inside of the dose-setting ring 140 to create an abutment surface 145a for the teeth 28 of the helical indentation 27 formed on the outer surface 25 of the plunger 20.

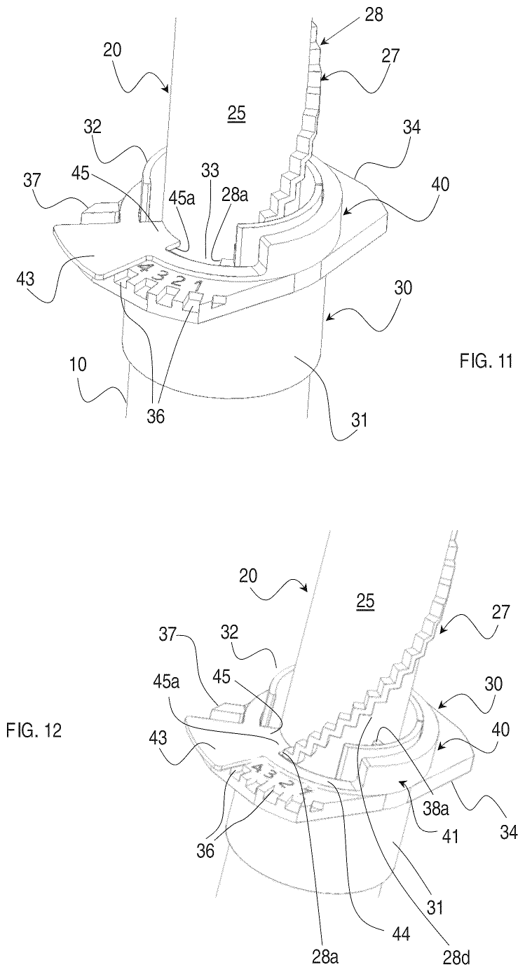

With reference to FIGS. 11 to 13, a method of multiple dosing a liquid or semiliquid product and subsequently administration of multiple doses, performed using the multi-dose syringe 100 according to the present invention will now be described. The dose-setting ring shown is the ring 40, described above and illustrated in FIGS. 7 and 8, but it is understood that a similar argument applies when using the dose-setting ring 140 illustrated in FIGS. 9 and 10.

Assume, for example, that the user wishes to administer a precise and repeated dose of the product equal to 5 ml. In this case, the user must, first of all, set the desired dose and, to this end, and as shown in FIG. 11, fit the tooth 42 of the ring 40 into the seat 36 marked with the number 5. At this point, the dose has been set and cannot be changed accidentally, so it can be administered repeatedly over time. In fact, and as set out above, an interference fit is established between the tooth 42 and the seat 36, so that the tooth 42 cannot be disengaged, unless intentionally. In addition, the elastic force due to the deformation of the dose-setting ring 40 keeps the tooth 42 pressed into the seat 36.

Once the dose to be administered has been set, the user, using the handle 26, rotates the plunger 20 about the longitudinal axis X thereof, until the longitudinal surface 28a of a tooth 28 of the helical indentation 27 abuts against the abutment surface 45a of the portion 45 of the tab 43 of the dose-setting ring 40. This operational condition is shown in FIG. 12. The rotation of the plunger 20 about the longitudinal axis X thereof is guaranteed by the presence of the connector 24 interposed between the end 21 of the plunger 20 and the rubber stopper 22. In other words, the connector 24, integral with the plunger 20, rotates inside the rubber stopper 22 stationary inside the hollow cylindrical body 10, when the user rotates the plunger 20.

At this point the set dose of product can be administered precisely and repeatably. To this end the user, if present, removes the cap 15 from the nozzle 13 of the hollow cylindrical body 10. Then, still using the handle 26, he presses the plunger 20 inside the hollow cylindrical body 10 until the second transverse surface 28d of a tooth 28 of the helical indentation 27 abuts against the stop element 38 and more precisely against the surface 38a of the flanged dose-setting sleeve 30. This causes the outlet of the dose of product previously set by the opening 14 of the nozzle 13. This operational condition is shown in FIG. 13.

It is now assumed that, at a later time, the user decides to dispense the same product dose previously set, to the same animal or to a different animal with the same weight or needing the same product dose.

In this case, the user merely has to repeat the above-described operations of rotating the plunger 20 about the longitudinal axis X thereof until the longitudinal surface 28a of a tooth 28 abuts against the abutment surface 45a of the tab 43, and of pressing the plunger 20 within the hollow cylindrical body 10 until the transverse surface 28d of a tooth 28 abuts against the stop element 38 and against the upper surface thereof 38a of the flanged sleeve 30.

As a result, if the dose to be administered does not vary, it is not necessary to move the dose-setting ring 40 to engage its teeth 42 in another seat 36 of the flanged sleeve 30, but to simply rotate the plunger 20. The amplitude of rotation of the plunger 20 then determines the size of the dose to be administered. In other words, if the dose does not vary, one gesture, i.e. rotating the plunger 20, is sufficient to set the dose to be administered. There is therefore no possibility of error, and the gesture to be performed is very simple. In addition, there are no external factors that can cause the set dose to vary, as the plunger 20 and the flanged sleeve 30 are held by the user and the ring 40 is locked in place on the flanged sleeve 30.

Otherwise, assume now that the user wishes to administer a precise, repeated dose over time of product different from that previously set on the dosing syringe 100, e.g. a dose of 2 ml.

In this case, he must release the ring 40 from the flanged sleeve 30 by grasping the tab 43 and lifting it up. This is facilitated by the fact that tab 43 is formed in the thinner, and therefore more flexible, portion 44 of the ring 40. The thickness of the portion 44 is preferably comparable to the thickness of tab 43.

Once the ring 40 has been released from the flanged sleeve 30, the user rotates the ring 40 with respect to the flanged sleeve 30 until at least one tooth 42 is at the seat 36 marked with the number 2, and then lowers the ring 40 so as to engage the tooth 42 in the seat 36.

Once the repeated dose to be dispensed has been set, the user, using the handle 26, rotates the plunger 20 about the longitudinal axis X thereof, until the longitudinal surface 28a of a tooth 28 of the helical indentation 27 abuts against the abutment surface 45a of the portion 45 of the tab 43 of the dose-setting ring 40.

Then, still using the handle 26, the user presses the plunger 20 inside the hollow cylindrical body 10 until the cross-sectional surface 28d of a tooth 28 abuts against an upper surface 38a of the stop element 38 of the flanged dose-setting sleeve 30. This causes the outlet of the dose of product previously set by the opening 14 of the nozzle 13.

Should the user wish to administer, on a one-off basis, a dose other than the preset one, he need not release the tooth 42 from the seat 36 in order to couple it to the seat corresponding to the new dose. Otherwise, the user can simply position a tooth 28 of the helical indentation in such a way that its two longitudinal surfaces 28a and 28b are in alignment, or substantial alignment, with the seat corresponding to the dose to be administered on a one-off basis. If the two longitudinal surfaces 28a and 28b do not align with a seat 36 of the flange 34, but are positioned between two seats 36, e.g. the seats 36 marked by the numbers 3 and 4, corresponding to a 3 ml dose and a 4 ml dose respectively, then the dose that is administered will be that of the seat 36 closest to the tooth 28. To administer the manually set dose, it is sufficient to press the plunger 20.

Finally, let us assume that a user wishes to use the multi-dose syringe 100 to administer many different doses, without the need to set a predetermined dose that can be repeated over time. In such a case, and as shown in detail in FIG. 14, it is sufficient to move the tab 43 of the ring 40, beyond the stop 37, so that the extension 45 is clamped between the stop and the side wall 33b of the opening 33 of the flanged dose-setting sleeve 30, and rotate the plunger 20 according to the animal to be treated, setting the desired dose using the numbers on the flange 34. In this case, the stop 37 and the side wall 33b of the opening 33 are spaced apart by an amount equal to accommodate the extension 45 of the tab 43 of the ring 40.

The aforementioned mode of operation, although less precise than the one involving the use of the ring for repeated doses, is more flexible in terms of dose choice, while still retaining the advantage of immediacy in dose preparation. In fact, a simple rotation of the plunger 20 is sufficient until the longitudinal surfaces 28a and 28b of any tooth 28 are aligned with a number on the flange 34 of the flanged dose-setting sleeve 30 and corresponding to a given dose. Once again, if the two longitudinal surfaces 28a and 28b do not align with a seat 36 of the flange 34, but are positioned between two seats 36, e.g. the seats 36 marked by the numbers 3 and 4, corresponding to a 3 ml dose and a 4 ml dose respectively, then the dose that is administered will be that of the seat 36 closest to the tooth 28. This is to ensure that there are no overdoses.

From the above description the features of the syringe and administration method of product doses according to the present invention, as well as the advantages thereof, are evident.

In particular, the multi-dose syringe and the method of multiple dosing a liquid or semiliquid product make it possible to prevent administration errors, since the user does not have to set the dose to be administered at each administration, thus being able, advantageously, to delegate the administration operation to a third party, without the need for special instructions. In addition, the multi-dose syringe prevents the animal causing dosing errors due to moving around. It is also noted that the multi-dose syringe according to the invention can also be used to administer one-off doses or different doses that are not repeated over time.

The invention thus conceived is susceptible to several modifications and variations, all falling within the scope of the inventive concept.

1-15. (canceled)

16. A multi-dose syringe comprising:
a hollow cylindrical body, containing a product to be administered in a dosed manner and from which a nozzle extends provided with a product outlet opening, and a plunger, sliding within the hollow cylindrical body;
a helical indentation is formed on an outer surface of the plunger, which comprises a plurality of teeth, each having a first and a second longitudinal surface and a first and a second transverse surface extending, parallel to each other, orthogonally from a respective longitudinal surface and in that it further comprises a flanged dose-setting sleeve, irremovably mounted on one end of the hollow cylindrical body for the entry of the plunger, and a dose-setting ring mounted on the flanged dose-setting sleeve and rotatable with respect thereto and with respect to the plunger,
wherein, on facing surfaces of the flanged sleeve and of the ring a series of seats and at least one tooth configured to be housed with interference in a respective seat are obtained, respectively, and wherein the plunger is rotatable with respect to the hollow cylindrical body until the first longitudinal surface of a tooth abuts against an abutment surface of the ring, projecting towards the inside of the hollow cylindrical body, so as to set a predetermined dose of the product to be administered.

17. The multi-dose syringe according to claim 16, wherein the flanged dose-setting sleeve comprises a first annular body for mounting the flanged sleeve on the end of the hollow cylindrical body for the entry of the plunger, a second annular body provided with an opening, and a flange interposed between the first annular body and the second annular body, the interference housing seats of the at least one tooth of the dose-setting ring being formed on a surface of the flange.

18. The multi-dose syringe according to claim 17, wherein on an inner surface of the second annular body there is a stop element, against which the first transverse surface of a tooth of the helical indentation abuts, so as to interrupt the stroke of the plunger within the hollow cylindrical body, when administering the dosed amount of product.

19. The multi-dose syringe according to claim 16, wherein the plunger comprises a rubber stopper associated with an end of the plunger intended to slide within the hollow cylindrical body, a connector being interposed between the rubber stopper and the end of the plunger, to allow a rotation of the plunger with respect to the rubber stopper within the hollow cylindrical body.

20. The multi-dose syringe according to claim 16, wherein the dose-setting ring comprises a main body, from which a tab extends outwards, the interference housing seats of the at least one tooth of the dose-setting ring being formed at a surface of the tab.

21. The multi-dose syringe according to claim 20, wherein the tab extends outwards from a portion of the dose-setting ring, which is thinner.

22. The multi-dose syringe according to claim 20, wherein the dose-setting ring comprises at least one, recess of localised weakening of the ring.

23. The multi-dose syringe according to claim 20, wherein the abutment surface a tooth of the helical indentation is formed on an extension of the tab facing towards the inside of the dose-setting ring.

24. The multi-dose syringe according to claim 17, wherein a stop extends from a surface of the flange facing the second annular body, adapted to limit the rotation of the dose-Preliminary setting ring with respect to the flanged dose-setting sleeve or to exclude the dose-setting ring from cooperation with the flanged dose-setting sleeve.

25. The multi-dose syringe according to claim 16, further comprising progressive numbers that are indicated at the seats of the flanged sleeve, one for each seat, each indicating an amount of product to be administered in a dosed manner.

26. The multi-dose syringe according to claim 16, wherein at another end of the plunger there is a handle having a faceted outer surface.

27. The multi-dose syringe according to claim 16, wherein the nozzle is formed in one piece with the hollow cylindrical body, or is a separate piece subsequently assembled on the hollow cylindrical body.

28. The method of multiple dosing a product performed using a multi-dose syringe according to claim 16, the method comprising the following steps:
rotation of a dose-setting ring with respect to a flanged dose-setting sleeve until at least one tooth of the ring is positioned at a respective seat of the flanged sleeve, corresponding to a predetermined volume of product to be dosed;
coupling with interference between the tooth and seat the; and
rotation of a plunger with respect to the flanged sleeve—ring assembly, until a first longitudinal surface of a tooth of a helical indentation formed on an outer surface of the plunger abuts against an abutment surface of the ring, projecting towards the inside of dose-setting ring, so as to set a predetermined dose of the product to be administered.

29. The method according to claim 28, further comprising, downstream of the rotating step of the plunger, a step of pressing the plunger towards the inside of the hollow cylindrical body, until a second surface of a tooth of the helical indentation abuts against a stop element formed in the flanged sleeve, so as to interrupt the stroke of the plunger inside the hollow cylindrical body, when the dosed amount of product is administered.

30. The method according to claim 28, further comprising the following additional steps of setting a new product dose:
lifting the ring from the flanged sleeve, so that the tooth is released from its seat;
rotation of the ring with respect to the flanged sleeve until at least one tooth of the ring is positioned at a respective seat of the flanged sleeve corresponding to a new dose to be administered;
coupling with interference between the tooth and seat the; and
rotation of a plunger with respect to the flanged sleeve—dose-setting ring assembly, until the first longitudinal surface of a tooth of a helical indentation of the plunger abuts against the abutment surface of the ring, projecting towards the inside of the dose-setting ring, so as to set a predetermined dose of the product to be administered.
